(19) United States
(12) Patent Application Publication
(10) Pub. No.: US 2025/0135104 A1
(43) Pub. Date: May 1, 2025

(54) FLOW CONTROL SYSTEMS AND METHODS FOR MEASURING FLOW RATES OF CANCER TREATMENT SYSTEMS

(71) Applicant: Boston Scientific Medical Device Limited, Galway Co. (IE)

(72) Inventors: Nachiket Gole, Pune (IN); Nitesh Ghananil Baviskar, Kalyan West (IN); Mihir Sukhatme, Mumbai (IN)

(21) Appl. No.: 18/926,372

(22) Filed: Oct. 25, 2024

Related U.S. Application Data
(60) Provisional application No. 63/546,334, filed on Oct. 30, 2023.

(51) Int. Cl. A61M 5/145

(52) U.S. Cl. CPC A61M 5/1452; A61M 2202/0007; A61M 2202/04; A61M 2205/3306; A61M 2205/3334; A61M 2205/3365; A61M 2205/583

(57) ABSTRACT

Embodiments herein relate to therapeutic fluid delivery devices with features to indicate and/or measure flow rate therefrom. In an embodiment, a therapeutic fluid delivery device is included having a barrel and a plunger. The plunger is configured to move within the barrel to expel a fluid from the barrel. The therapeutic fluid delivery device can include a rotatable element, wherein movement of the plunger relative to the barrel causes the rotatable element to rotate. The therapeutic fluid delivery device can be configured to measure a rotation speed of the rotatable element. Other embodiments are also included herein.

This application claims the benefit of U.S. Provisional Application No. 63/546,334, filed Oct. 30, 2023, the content of which is herein incorporated by reference in its entirety.

### FIELD

Embodiments herein relate to therapeutic fluid delivery devices with features to indicate and/or measure flow rate therefrom.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division. Surgery is a common first-line therapy for many cancerous tumors. However, not every tumor can be surgically removed.

### SUMMARY

Embodiments herein relate to therapeutic fluid delivery devices with features to indicate and/or measure flow rate therefrom. In a first aspect, a therapeutic fluid delivery device can be included having a barrel and a plunger. The plunger can be configured to move within the barrel to expel a fluid from the barrel. The device can also include a rotatable element. Movement of the plunger relative to the barrel can cause the rotatable element to rotate. The therapeutic fluid delivery device can be configured to measure a rotation speed of the rotatable element.

In a second aspect, in addition to one or more of the preceding or following aspects, or in the alternative to some aspects, the therapeutic fluid delivery device can be configured to calculate a fluid flow rate based on the rotation speed.

In a third aspect, in addition to one or more of the preceding or following aspects, or in the alternative to some aspects, the therapeutic fluid delivery device can be configured to output the fluid flow rate to a device user.

In a fourth aspect, in addition to one or more of the preceding or following aspects, or in the alternative to some aspects, the therapeutic fluid delivery device can be configured to generate a signal including the rotation speed of the rotatable element and/or the calculated fluid flow rate.

In a fifth aspect, in addition to one or more of the preceding or following aspects, or in the alternative to some aspects, the rotatable element can include a pinion gear.

In a sixth aspect, in addition to one or more of the preceding or following aspects, or in the alternative to some aspects, the device can further include a rack, wherein the rack can be attached to the plunger, and wherein the rack engages with a pinion gear and causes the same to rotate as the plunger is depressed.

In a seventh aspect, in addition to one or more of the preceding or following aspects, or in the alternative to some aspects, the rotatable element can include a wheel.

In an eighth aspect, in addition to one or more of the preceding or following aspects, or in the alternative to some aspects, the therapeutic fluid delivery device can be configured to measure the rotation speed of the rotatable element using a tachometer or an optical sensor.

In a ninth aspect, in addition to one or more of the preceding or following aspects, or in the alternative to some aspects, the therapeutic fluid delivery device can further include an orifice, wherein the orifice can be in fluid communication with the barrel, and a fluid supply tube, wherein the fluid supply tube can be in fluid communication with the orifice.

In a tenth aspect, a therapeutic fluid delivery device can be included having a barrel and a plunger. The plunger can be configured to move within the barrel to expel a fluid from the barrel. A rotatable element can also be included, wherein movement of the plunger relative to the barrel causes the rotatable element to rotate and wherein the therapeutic fluid delivery device can be configured to visibly indicate the rotation speed of the rotatable element.

In an eleventh aspect, in addition to one or more of the preceding or following aspects, or in the alternative to some aspects, the rotatable element can include a pinion gear.

In a twelfth aspect, in addition to one or more of the preceding or following aspects, or in the alternative to some aspects, further can include a rack, wherein the rack can be attached to the plunger, and wherein the rack engages with the pinion gear and causes the same to rotate as the plunger can be depressed.

In a thirteenth aspect, in addition to one or more of the preceding or following aspects, or in the alternative to some aspects, the rotatable element can include a wheel.

In a fourteenth aspect, in addition to one or more of the preceding or following aspects, or in the alternative to some aspects, the therapeutic fluid delivery device can be configured to visibly indicate the rotation speed of the rotatable element using a mechanical mechanism.

In a fifteenth aspect, in addition to one or more of the preceding or following aspects, or in the alternative to some aspects, the therapeutic fluid delivery device can be configured to visibly indicate the rotation speed of the rotatable element using a centrifugal indicator assembly.

In a sixteenth aspect, in addition to one or more of the preceding or following aspects, or in the alternative to some aspects, the centrifugal indicator assembly can include opposed weights, opposed hinges, and a shaft. When the weights rotate around the shaft centrifugal force causes the hinges to bend and the weights to move radially outward from the shaft with increased rotational speed.

In a seventeenth aspect, a method of measuring fluid flow rate of a fluid delivery device can be included. The method can include moving a plunger within a barrel to expel a fluid from the barrel and causing a rotatable element connected to the barrel to rotate. The method can further include measuring a rotation speed of the rotatable element and calculating a fluid flow rate based on the rotation speed.

In an eighteenth aspect, in addition to one or more of the preceding or following aspects, or in the alternative to some aspects, the method can further include outputting the fluid flow rate to a device user.

In a nineteenth aspect, in addition to one or more of the preceding or following aspects, or in the alternative to some aspects, the method can further include generating a signal including the rotation speed of the rotatable element and/or the calculated fluid flow rate.

In a twentieth aspect, in addition to one or more of the preceding or following aspects, or in the alternative to some aspects, the method can further include measuring the rotation speed of the rotatable element using a tachometer or an optical sensor.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a diagram of a cancer treatment system in accordance with various embodiments herein.

FIG. 2 is a schematic view of a therapeutic fluid delivery device in accordance with various embodiments herein.

FIG. 3 is a schematic view of a therapeutic fluid delivery device in accordance with various embodiments herein.

FIG. 4 is a schematic view of a therapeutic fluid delivery device in accordance with various embodiments herein.

FIG. 5 is a schematic view of a therapeutic fluid delivery device in accordance with various embodiments herein.

FIG. 6 is a schematic view of a therapeutic fluid delivery device in accordance with various embodiments herein.

FIG. 7 is a schematic view of a therapeutic fluid delivery device in accordance with various embodiments herein.

FIG. 8 is a schematic view of a therapeutic fluid delivery device in accordance with various embodiments herein.

FIG. 9 is a schematic view of a therapeutic fluid delivery device in accordance with various embodiments herein.

FIG. 10 is a flowchart of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, surgery is a common first-line therapy for many cancerous tumors. However, not every tumor can be surgically removed. Another approach to treating some types of cancer can include delivering radiation to cancerous tissue to destroy cancerous cells therein. In one approach, microspheres such as small glass microspheres that have Y-90 mixed into the glass can be deposited in or near a cancerous tumor and beta radiation emitting therefrom can damage DNA of the cancerous cells inducing apoptosis and cell cycle arrest.

Delivery of microspheres can be performed using a catheter, such as a microcatheter placed in vasculature leading to a tumor, such as an artery supplying blood to the tumor. The microspheres can be combined with a fluid, such as saline, to form a suspension that is then directed through the catheter to the site of deposition.

In some approaches, a clinician must utilize a syringe or similar device to deliver the fluid to be mixed with the microspheres. The clinician advances the plunger of the syringe at a desired pressure/speed resulting in a particular flow rate of fluid through the system for a particular amount of time to a achieve a target volume of suspension delivered out of the catheter and into the patient. However, it is tedious for system users to manage aspects of this process. For example, it is tedious and difficult for system users to consistently apply pressure to the syringe to achieve a desired speed of plunger depression and resulting flow rate of fluid (such as between 5 ml/min and 20 ml/min, or within other flow rate bounds). It is also challenging to accurately determine the actual flow rate achieved.

Systems and methods herein can be used to accurately measure flow rates of fluid from fluid delivery devices. Such flow rates can be measured without making direct contact with fluids so that sterility can be maintained. In some embodiments, a therapeutic fluid delivery device includes a rotatable element (such as a pinion gear, wheel, or the like) that can be caused to rotate when a plunger or similar mechanism of a fluid delivery device is depressed. For example, movement of the plunger relative to a barrel of a fluid delivery device can cause the rotatable element to rotate. Thus, the rotation speed of the rotatable element can stand in a fixed relationship to the speed of plunger depression. The rotation speed can be determined in various ways using various sensors as described further below. In some embodiments, the flow rate of fluid being ejected from the therapeutic fluid delivery device can be actively calculated based on knowledge of the speed of plunger depression (as determined by sensing rotatable element rotation speed and the fixed relationship between rotatable element rotation speed and speed of plunger depression) and the cross-sectional area of the barrel of the fluid delivery device (e.g., distance/minute X cross-sectional area=volume/minute). Such calculated values of flow rate (such as in ml/min or presented in other units) can be displayed to a system user (to assist them in actuating the plunger to provide a desired flow rate) and/or be provided in a signal that can be transmitted via wired or wireless techniques to another device for use and/or display. However, in some embodiments, the flow rate of fluid being ejected from the therapeutic fluid delivery device can be passively indicated to a system user (such as via a mechanical mechanism, examples of which are provided herein) to assist them in actuating the plunger to provide a desired flow rate.

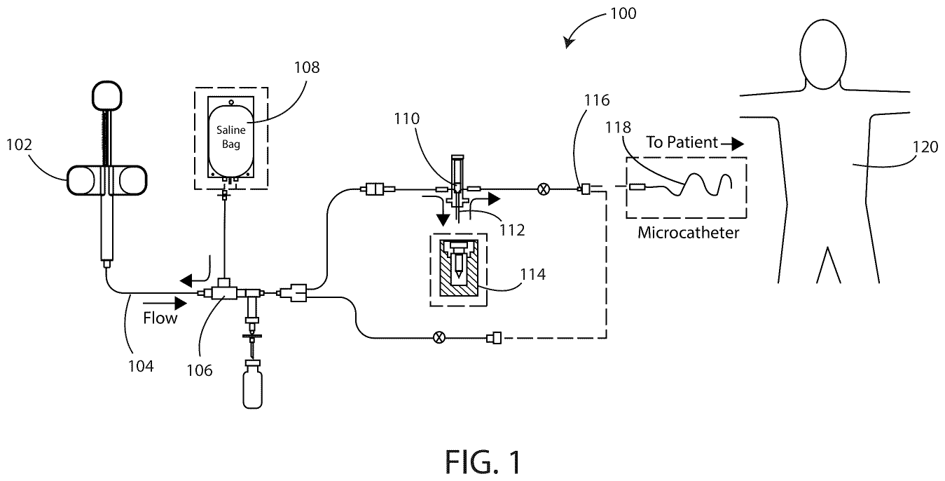

Referring now to FIG. 1, a diagram of a cancer treatment system 100 is shown in accordance with various embodiments herein. Major parts of the cancer treatment system 100 include a therapeutic fluid delivery device 102, a fluid supply tube 104, and a flow control valve 106. The cancer treatment system 100 also includes a saline supply reservoir 108. The cancer treatment system 100 also includes a fluid injection and suspension withdrawal device 110. The cancer treatment system 100 also includes a radioactive microsphere supply reservoir 114. The cancer treatment system 100 also includes an outflow port 116. The cancer treatment system 100 also includes a microcatheter 118. FIG. 1 shows a patient 120 in which the microcatheter 118 can be inserted to deliver the therapeutic suspension of microspheres.

In use, (omitting some possible operations for ease of explanation) the clinician or other system user can pull back on a plunger or similar mechanism of therapeutic fluid delivery device 102 causing fluid (such as saline) to be withdrawn from the saline supply reservoir 108, through the flow control valve 106 and the fluid supply tube 104, and into the fluid delivery device 102. Then the clinician or other system user can depress the plunger causing fluid to flow from the therapeutic fluid delivery device 102, through the fluid supply tube 104, through the flow control valve 106, and into the fluid injection and suspension withdrawal device 110. The fluid injection and suspension withdrawal device 110 can be in fluid communication with the radioactive microsphere supply reservoir 114 and can direct a flow of fluid into the radioactive microsphere supply reservoir 114 coming from the therapeutic fluid delivery device 102 such as through one of a pair of needles, cannulas, or tubes 112. The fluid can become mixed with microspheres in the radioactive microsphere supply reservoir 114 forming a suspension which can then exit via the fluid injection and suspension withdrawal device 110 via another needle, cannula, or tube 112 and through tubing and out of the outflow port 116 and into the microcatheter 118 and into a desired site of the patient 120. It will be appreciated that various other operations can also be performed including, but not limited to, system priming, bubble removal, one or more flushing operations, and the like.

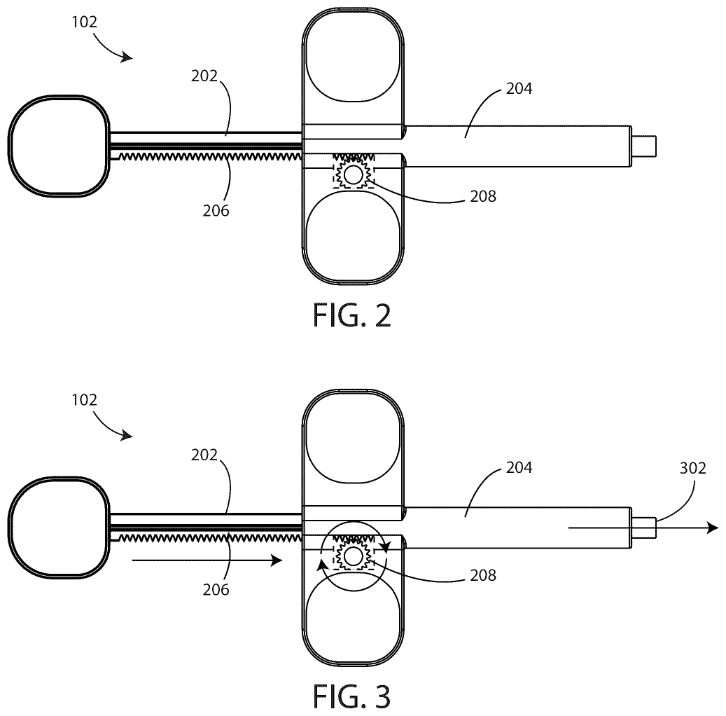

Referring now to FIG. 2, a schematic view of a therapeutic fluid delivery device 102 is shown in accordance with various embodiments herein. In this example, the therapeutic fluid delivery device 102 includes a plunger 202 and a barrel 204. The therapeutic fluid delivery device 102 also includes a rack 206 and a rotatable element 208. The rack 206 can include teeth thereon. The rack 206 can be in a fixed relationship with the plunger 202. The teeth of the rack 206 can engage with teeth on the rotatable element 208 (such in the case of a pinion gear). In this case, the rotatable element 208 can be attached (directly or indirectly) to a portion of the barrel or a structure connected thereto. Notably, the rotatable element 208 is not in fluid communication with an interior portion of the barrel 204, allowing fluid within the interior portion of the barrel 204 to remain sterile. It will be appreciated that parts of the therapeutic fluid delivery device 102 can be formed of various materials including polymers, composites, metals, glasses, and the like.

In various embodiments, the plunger 202 can be configured to move within a barrel 204 to expel a fluid from the barrel 204, such as the case when a plunger of a syringe into pushed into the barrel of the syringe. In various embodiments, the rack 206 engages with a pinion gear and causes the same to rotate as a plunger 202 is depressed. Referring now to FIG. 3, the therapeutic fluid delivery device 102 can again include a plunger 202, a barrel 204, a rack 206, and a rotatable element 208. In operation, the plunger 202 can be pushed into the barrel 204 in the direction of the arrow shown. This causes fluid to be expelled from the barrel 204 and out the orifice 302. The movement of the plunger 202 relative to the barrel 204 causes the rotatable element 208 to rotate in the direction shown when the plunger 202 is being depressed. In various embodiments, the therapeutic fluid delivery device 102 can then be configured to calculate a fluid flow rate based on the rotation speed.

While in some embodiments the rotatable element 208 can take the form of a pinion gear, it will be appreciated that it can also take on other forms as well. For example, the rotatable element 208 can take the form of a wheel, a shaft, or the like.

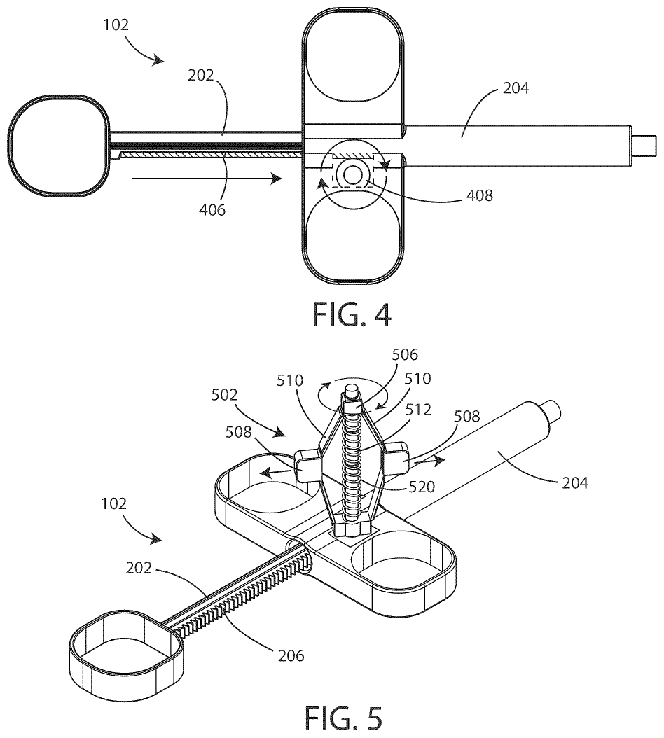

Referring now to FIG. 4, a schematic view of a therapeutic fluid delivery device 102 is shown in accordance with various embodiments herein. As before, therapeutic fluid delivery device 102 includes a plunger 202 and a barrel 204. However, in this embodiment, the therapeutic fluid delivery device 102 includes a wheel contact mechanism 406 and the rotatable element takes the form of a wheel 408. Instead of relying upon the interface between teeth on the rack with teeth on a pinion gear to cause rotation of the rotatable element, in this embodiment friction between the wheel contact mechanism 406 and the wheel 408 causes the wheel 408 to rotate as the plunger 202 is depressed.

In some embodiments, it can be valuable to visually indicate (directly or some type of proxy of the same) a rotation speed. While this can be done by displaying information on a display screen, in some cases it can be useful to accomplish this using only mechanical means and no electric power. Referring now to FIG. 5, a schematic view of a therapeutic fluid delivery device 102 is shown in accordance with various embodiments herein. As before, the therapeutic fluid delivery device 102 includes a plunger 202, a barrel 204, a rack 206, and a rotatable element (not visible in FIG. 5). FIG. 5 also shows a centrifugal indicator assembly 502. The centrifugal indicator assembly 502 includes an indicator hub 506, opposed weights 508, opposed hinges 510, a shaft 512, and spring 520. In operation, there is a linkage between the rotatable element and the shaft 512. When the shaft is not rotating, the force provided by the spring 520 keeps the indicator hub 506 at a position (the zero position) towards the distal end of the shaft 512. As the shaft 512 rotates, centrifugal force acts upon the opposed weights 508 pushing them outward radially and causing the opposed hinges 510 to flex. The weights 508 move radially outward from the shaft 512 with increased rotational speed generating increased centrifugal force. This causes the indicator hub 506 to push against the spring 520 and move down the shaft 512 in the direction of the rotatable element 208. The spring 520 maintains the indicator hub 506 in a position of equilibrium for a given constant rotation speed. The shaft 512 can include various indicia thereon related to rotation speeds and, in turn, flow rates. The indicia at the point where the indicator hub 506 lines up with the shaft can indicate the rotation speed and/or the flow rate. In this manner, the therapeutic fluid delivery device 102 can be configured to visibly indicate the rotation speed of the rotatable element 208. For example, the therapeutic fluid delivery device 102 can be configured to visibly indicate the rotation speed of the rotatable element 208 using a mechanical mechanism.

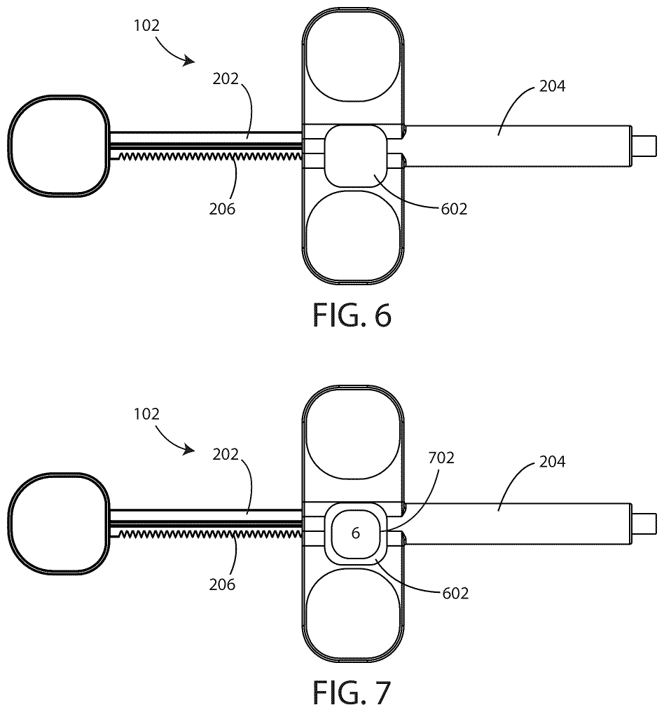

Rotational speed of the rotatable element 208 can be actively sensed in various ways. Referring now to FIG. 6, a schematic view of a therapeutic fluid delivery device 102 is shown in accordance with various embodiments herein. As before, the therapeutic fluid delivery device 102 includes a plunger 202, a barrel 204, a rack 206, and a rotatable element 208. In this embodiment, the therapeutic fluid delivery device 102 also includes a rotation sensor 602. In some embodiments, the rotation sensor 602 can take the form of a tachometer (mechanical or electronic), an optical sensor (such as one detecting changes in reflected or transmitted light intensity), a magnetic sensor, an acoustic sensor, or the like. However, many different rotation sensors can be utilized herein taking advantage of mechanical, electrical, magnetic, acoustic, and/or optical principles. In some embodiments, the rotation sensor 602 can be in electronic communication with control circuitry (not shown in this view). The control circuitry can include various components to execute operations herein including one or more of a microprocessor, an application specific integrated circuit (ASIC), memory circuit (such as random access memory (RAM), read only memory (ROM)) and/or Electrically Erasable ROM (EEROM/Flash), recorder circuitry, controller circuit, a telemetry circuit, a power supply circuit (including, for example, a battery), a timing circuit, and, a recharging circuit, amongst others components. The control circuit can be configured to receive a signal from the rotation sensor 602. In various embodiments, the control circuitry can perform calculations herein, such as calculating a flow rate based on a rotation speed of the rotatable element. A fixed relationship (or conversion factor) can exist between rotation speed of the rotatable element and a flow rate and the conversion factor can be stored by the device and/or received through user input. In some embodiments, the control circuit can use a conversion factor to convert rotation speed of the rotatable element into a calculated flow rate.

In some embodiments, the rotation speed, calculated flow rate, and/or other information can be visually displayed to the system user. Referring now to FIG. 7, a schematic view of a therapeutic fluid delivery device 102 is shown in accordance with various embodiments herein. As before, the therapeutic fluid delivery device 102 includes a plunger 202, a barrel 204, a rack 206, a rotatable element 208, and a rotation sensor 602. In this embodiment, the therapeutic fluid delivery device 102 also includes a display device 702. The display device 702 can include an LCD screen or other display device component. Various pieces of information can be displayed on the display device 702 including, but not limited to, rotation speed, actual flow rate, target flow rate, delivery time, and the like. Thus, the therapeutic fluid delivery device 102 can be configured to output the fluid flow rate and/or other information to a device user.

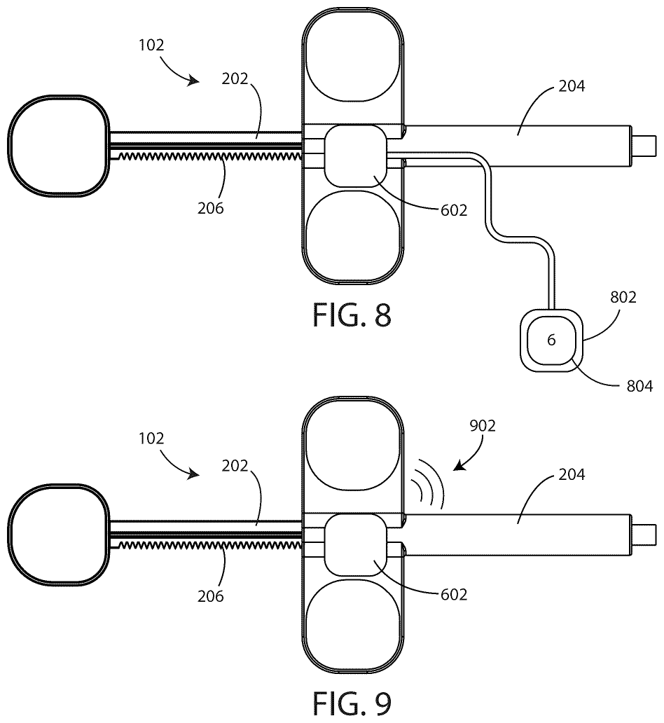

In some embodiments, signals and/or data relating to delivery of the fluid/suspension can be transmitted by the therapeutic fluid delivery device 102. Referring now to FIG. 8, a schematic view of a therapeutic fluid delivery device 102 is shown in accordance with various embodiments herein. As before, the therapeutic fluid delivery device 102 includes a plunger 202, a barrel 204, a rack 206, a rotatable element 208, and a rotation sensor 602. In this embodiment, the therapeutic fluid delivery device 102 also includes an external display device 802. Signals can be transmitted to external display device 802 which can include a display screen 804 thereon. However, it will be appreciated that signals/data can also be transmitted wirelessly. Referring now to FIG. 9, a schematic view of a therapeutic fluid delivery device 102 is shown in accordance with various embodiments herein. As before, the therapeutic fluid delivery device 102 includes a plunger 202, a barrel 204, a rack 206, a rotatable element 208, and a rotation sensor 602. The therapeutic fluid delivery device 102 is illustrated sending a wireless signal 902 that can include information related to fluid/suspension delivery as described herein.

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

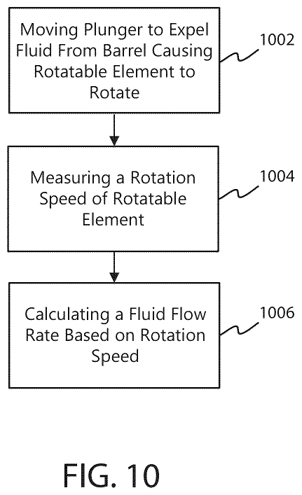

In an embodiment, a method of measuring fluid flow rate of a fluid delivery device is included. Referring now to FIG. 10, a flowchart of a method is shown in accordance with various embodiments herein. The method of measuring fluid flow rate of a fluid delivery device includes moving a plunger within a barrel to expel a fluid from the barrel and causing a rotatable element connected to the barrel to rotate 1002. The method of measuring fluid flow rate of a fluid delivery device also includes measuring a rotation speed of the rotatable element 1004. The method of measuring fluid flow rate of a fluid delivery device also includes calculating a fluid flow rate based on the rotation speed 1006.

In various embodiments, the method can further include outputting the fluid flow rate to a device user. In various embodiments, further can include generating a signal including the rotation speed of the rotatable element and/or the calculated fluid flow rate. In various embodiments, further can include measuring the rotation speed of the rotatable element using a tachometer or an optical sensor.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A therapeutic fluid delivery device comprising:
a barrel;
a plunger, wherein the plunger is configured to move within the barrel to expel a fluid from the barrel; and
a rotatable element;
wherein movement of the plunger relative to the barrel causes the rotatable element to rotate; and
wherein the therapeutic fluid delivery device is configured to measure a rotation speed of the rotatable element.

2. The therapeutic fluid delivery device of claim 1, wherein the therapeutic fluid delivery device is configured to calculate a fluid flow rate based on the rotation speed.

3. The therapeutic fluid delivery device of claim 2, wherein the therapeutic fluid delivery device is configured to output the fluid flow rate to a device user.

4. The therapeutic fluid delivery device of claim 2, wherein the therapeutic fluid delivery device is configured to generate a signal including the rotation speed of the rotatable element and/or the calculated fluid flow rate.

5. The therapeutic fluid delivery device of claim 1, the rotatable element comprising a pinion gear.

6. The therapeutic fluid delivery device of claim 5, further comprising a rack;
wherein the rack is attached to the plunger; and
wherein the rack engages with the pinion gear and causes the same to rotate as the plunger is depressed.

7. The therapeutic fluid delivery device of claim 1, the rotatable element comprising a wheel.

8. The therapeutic fluid delivery device of claim 1, wherein the therapeutic fluid delivery device is configured to measure the rotation speed of the rotatable element using a tachometer or an optical sensor.

9. The therapeutic fluid delivery device of claim 1, further comprising:
an orifice, wherein the orifice is in fluid communication with the barrel; and
a fluid supply tube, wherein the fluid supply tube is in fluid communication with the orifice.

10. A therapeutic fluid delivery device comprising:
a barrel;
a plunger, wherein the plunger is configured to move within the barrel to expel a fluid from the barrel; and
a rotatable element;
wherein movement of the plunger relative to the barrel causes the rotatable element to rotate; and
wherein the therapeutic fluid delivery device is configured to visibly indicate the rotation speed of the rotatable element.

11. The therapeutic fluid delivery device of claim 10, the rotatable element comprising a pinion gear.

12. The therapeutic fluid delivery device of claim 11, further comprising a rack;
wherein the rack is attached to the plunger; and
wherein the rack engages with the pinion gear and causes the same to rotate as the plunger is depressed.

13. The therapeutic fluid delivery device of claim 10, the rotatable element comprising a wheel.

14. The therapeutic fluid delivery device of claim 10, wherein the therapeutic fluid delivery device is configured to visibly indicate the rotation speed of the rotatable element using a mechanical mechanism.

15. The therapeutic fluid delivery device of claim 10, wherein the therapeutic fluid delivery device is configured to visibly indicate the rotation speed of the rotatable element using a centrifugal indicator assembly.

16. The therapeutic fluid delivery device of claim 15, the centrifugal indicator assembly comprising:
opposed weights;
opposed hinges; and
a shaft; and
wherein the weights rotate around the shaft generating centrifugal force causing the hinges to bend and the weights to move radially outward from the shaft with increased rotational speed.

17. A method of measuring fluid flow rate of a fluid delivery device comprising:
moving a plunger within a barrel to expel a fluid from the barrel and causing a rotatable element connected to the barrel to rotate;
measuring a rotation speed of the rotatable element; and
calculating a fluid flow rate based on the rotation speed.

18. The method of claim 17, further comprising outputting the fluid flow rate to a device user.

19. The method of claim 17, further comprising generating a signal including the rotation speed of the rotatable element and/or the calculated fluid flow rate.

20. The method of claim 17, further comprising measuring the rotation speed of the rotatable element using a tachometer or an optical sensor
